(19) United States
(12) Patent Application Publication
(10) Pub. No.: US 2012/0283602 A1
(43) Pub. Date: Nov. 8, 2012

(54) WIRELESS ELECTRONIC PEGBOARD SETUP FOR QUANTIFICATION OF DEXTERITY

(75) Inventors: Steven E. Irby, Houston, TX (US); Dillon P. Eng, Seattle, WA (US); Rachel Jackson, Springfield, VA (US); Allison C. Scully, St. Louis, MO (US); Jessica Scully, St. Louis, MO (US); Robert Likamwa, Bellevue, WA (US); Marcia K. O'Malley, Houston, TX (US); Z. Maria Oden, Houston, TX (US); Gloria R. Gogola, Houston, TX (US); Avery Laurel Cate, Inez, TX (US)

(73) Assignees: Shriners Hospital for Children, Tampa, FL (US); William Marsh Rice University, Houston, TX (US)

(21) Appl. No.: 13/446,610

(22) Filed: Apr. 13, 2012

Related U.S. Application Data
(60) Provisional application No. 61/475,079, filed on Apr. 13, 2011.

(51) Int. Cl. A61B 5/11

(52) U.S. Cl. 600/595

(57) ABSTRACT

An electronic pegboard setup is provided for assessing patient dexterity. In certain embodiments, the pegboard setup may be wireless and may employ pegs equipped with sensors that that allow tracking of the motion of the peg in three-dimensions and over time. In one such embodiment, a visual output of the motion path may be provided to the clinician. In such an embodiment, the device may provide quantitative data regarding motion path to assess patient dexterity.

### CROSS-REFERENCE TO RELATED APPLICATIONS

This application claims the benefit of U.S. Provisional Patent Application No. 61/475,079, entitled “WIRELESS ELECTRONIC PEGBOARD SETUP FOR QUANTIFICATION OF DEXTERITY,” and filed Apr. 13, 2011, which is herein incorporated by reference in its entirety for all purposes.

### BACKGROUND

The present disclosure relates generally to the quantification of dexterity in people with motor impairment.

Currently, to assess patient dexterity, such as in cerebral palsy patients, trained clinicians to spend hours of time watching video of the patients completing dexterous tasks. Further, the patients are required to spend several hours completing these dexterous tasks, which is both tiresome and boring. In addition, to perform the tests, the patients may be required to go to a specific room for testing. Such testing and assessment may be less than ideal in practice.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

An electronic pegboard setup is provided for assessing patient dexterity. In certain embodiments, the pegboard setup may be wireless and may employ pegs equipped with sensors that that allow tracking of the motion of the peg in three-dimensions and over time. In one such embodiment, a visual output of the motion path may be provided to the clinician. In such an embodiment, the device may provide quantitative data regarding motion path to assess patient dexterity.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a dexterity measurement system, in accordance with aspects of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, the calculations, correlations, and/or measurements discussed herein may be performed using one or more suitable computer-implemented algorithms, such as may be stored on a computer-readable memory or medium for execution by a suitable processing component in communication with the medium or memory. In addition, previously determined constants or correlations may be stored on the computer-readable memory or medium and accessed by the processing component during execution of the algorithms. The processor may also receive inputs or measurements from a measurement device, such as a spectrophotometer, or from personnel. Such inputs may be processed used as inputs to the algorithms during execution of the algorithms. Examples of devices having suitable processing components, memory components, user interface circuitry, and/or circuitry for accessing local or remote media include, but are not limited to, desktop, notebook, and tablet computers, personal digital assistants, cellular telephones, media players, and so forth.

The purpose of the presently disclosed electronic pegboard setup is to aid physicians and clinicians in quantifying dexterity of cerebral palsy patients or other motion-impaired patients, and examining changes and improvements in dexterity during and after treatments, such as surgery or therapy. This device solves the problem of requiring trained clinicians to spend hours of time watching video of cerebral palsy patients completing dexterous tasks in order to assess patient dexterity.

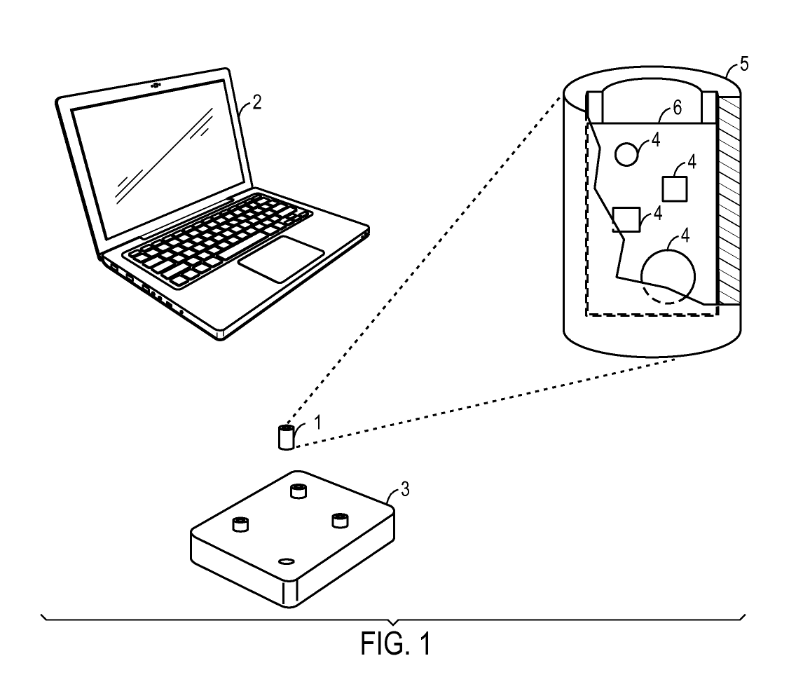

Turning to FIG. 1, in one embodiment, the device will consist of a pegboard 3 with nine or sixteen holes and nine or sixteen corresponding pegs 1, though other numbers of pegs 1 and/or holes (such as one through sixteen) may also be employed. In one implementation, the pegs 1 are 10 mm to 150 mm high (such as 60 mm high) and 5 mm to 70 mm in diameter (such as 35 mm in diameter), and each hole is slightly wider in diameter to accommodate the pegs 1, and is deep enough so that the peg 1 is available to grab (such as 1 cm, 2 cm, or 3 cm protruding). Further, the dimensions of the pegs 1 and pegboard 3 can be altered to accommodate patients who have trouble removing the pegs 1 from the board 3.

Each of the pegs 1 will be equipped (see peg cutaway view 5) with sensors, such as a tri-axial accelerometer, gyroscope, and microcontroller, to track the motion of the peg 1 in three-dimensions over time. In one implementation, two coin cell batteries and a power regulator will power the electronics 4 in each peg 1. In such an implementation, the pegs 1 are powered individually because the pegs 1 are removable from the pegboard 3, and the entire board 3 may be portable and wireless. The microcontroller may communicate acceleration and orientation data to an outside computer 2 via a USB device using a radio-frequency signal. A Matlab routine or other suitable generated code may be used to analyze the raw data and output a three-dimensional graph of position with respect to time. A novel feature of the device is that it will track a three-dimensional motion path without the aid of a video camera.

Other types of output data may also be provided. For example, instead of providing position data, the device could provide actual acceleration data, peak velocity, or even jerk, the derivative of acceleration.

In one implementation, the electronics of the device, including the accelerometer, gyroscope and microcontroller, may first be wired together on a suitable printed circuit board 6. These electronics 4 will then be inserted into the respective peg 1. Each peg 1 will be hollowed out, and the electronics will fit appropriately into the peg 1. The lid of the peg 1 will press fit into the body of the peg 1. The pegs 1 may be made out of acrylonitrile butadiene styrene (ABS) plastic, polyurethane coated wood, or other suitable materials, and the pegboard 3 may be made out of PVC, polyurethane coated wood, or other suitable materials. In one embodiment, the pegs 1 may be printed on a 3D printer and the pegboard 3 may be machined to the appropriate size and shape. The microcontroller and USB transceiver may be programmed to communicate data that is collected from the peg 1 to a computer 2. A LabVIEW routine or other routine may be written to collect data in real time that is transmitted to the computer 2, and a MATLAB routine or other post-processing scheme may be used to process the raw acceleration data into position data and plot this in three dimensions.

It may be desirable that the starting and ending points of the pegs 1 be known exactly to reduce drift in position data. A software scheme can be applied to the post-processing in MATLAB in order to reduce drift in position. No calibration should be necessary at the start of each test due to the use of this post-processing scheme. A way to accurately obtain position data is through the use of cameras and image-processing.

The device may be portable and wireless, allowing for less setup and more frequent testing throughout hospitals. By making the device a wireless pegboard setup, which communicates via radio frequency, clinicians can go to the rooms of the patients instead of the patients going to a specific room for testing. In addition, a testing session on the device may be completed in five minutes or less.

A visual output of the motion path will be provided to the clinician in near real-time, e.g., in minutes or immediately, due to the communication between the pegboard 3 and a computer 2. The device will provide quantitative data regarding motion path to assess patient dexterity, an improvement over the qualitative assessment that comes from a physician watching a video. This will allow clinicians to assess dexterity quantitatively and efficiently. This device could also be used to quantify dexterity for people with motor control impairments due to syndromes other than cerebral palsy, or be used to assess motor skill development in children.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art, including combinations of aspects or features of the embodiments and examples disclosed herein. Such other examples are intended to be within the scope of the claims if they have structural elements that do not differ from the literal language of the claims, or if they include equivalent structural elements with insubstantial differences from the literal languages of the claims. The specific embodiments described above have been shown by way of example, and it should be understood that these embodiments may be susceptible to various modifications and alternative forms, including combinations of various features and aspects of the examples or embodiments discussed herein. It should be further understood that the claims are not intended to be limited to the particular forms disclosed, but rather to cover all modifications, equivalents, and alternatives falling within the spirit and scope of this disclosure.

1. A system for measuring dexterity, comprising:
a plurality of pegs, each peg comprising a plurality of sensors suitable for tracking the position of the respective peg in three-dimensions and in time; and
a pegboard comprising a plurality of holes, each hole sized to accommodate a peg of the plurality of pegs.

2. The system of claim 1, wherein the pegs comprise one or more batteries powering the respective peg.

3. The system of claim 1, wherein the pegs are approximately 49 mm high and approximately 28 mm in diameter.

4. The system of claim 1, wherein the pegs are each equipped with a tri-axial accelerometer, a gyroscope, and a microcontroller.

5. The system of claim 4, wherein the microcontroller communicates acceleration and orientation data to an external computer.

6. The system of claim 5, wherein the acceleration and orientation data is used to generate a three-dimensional graph of position with respect to time on the external computer.

7. The system of claim 1, wherein the pegboard is portable.

8. The system of claim 1, wherein the system is configured to communicate wirelessly.

9. A method for assessing dexterity, comprising the acts of:
collecting three-dimensional data over time generated by a patient placing and moving pegs within a pegboard, wherein the three-dimensional data is generated by one or more sensors provided in the pegs;
generating a three-dimensional graph of position with respect to time based on the three-dimensional data; and
analyzing the three-dimensional graph to assess the dexterity of a patient.

10. The method of claim 9, wherein the act of collecting the three-dimensional data over time takes less than five minutes.

11. The method of claim 9, comprising taking the pegboard and pegs to a room of the patient.

12. The method of claim 9, comprising wirelessly transmitting the three-dimensional data to a computer that generates the three-dimensional graph.

13. A peg for use in a dexterity measuring system, the peg comprising:
a tri-axial accelerometer configured to generate acceleration data in three-dimensions over time;
a gyroscope configured to generate orientation data over time; and
a microcontroller configured to wirelessly communicate the acceleration data and the orientation data.

14. The peg of claim 13, wherein the peg further comprises:
a power regulator; and
one or more batteries;
wherein the power regulator and the one or more batteries, power the operation of the peg.

15. The peg of claim 14, wherein the one or more batteries comprise two coin cell batteries.

16. The peg of claim 13, comprising a printed circuit board to which the tri-axial accelerometer, the gyroscope, and the microcontroller are connected.

17. The peg of claim 13, wherein the peg is constructed from acrylonitrile butadiene styrene.

18. A pegboard, comprising:
a plurality of holes, each sized to accommodate a peg;
wherein the holes provide exact starting an ending points for the movement of the pegs.

19. The pegboard of claim 18, wherein the pegboard is constructed from polyvinyl chloride.

20. The pegboard of claim 18, wherein the plurality of holes comprises nine or sixteen holes.
